(19) United States
(12) Patent Application Publication
(10) Pub. No.: US 2008/0287748 A1
(43) Pub. Date: Nov. 20, 2008

(54) System and method for physiological data readings, transmission and presentation

(76) Inventors: Demetrios Sapounas, Leesburg, VA (US); Emil Jovanov, Huntsville, AL (US)

Correspondence Address: ARENT FOX LLP, 1050 CONNECTICUT AVENUE, N.W., SUITE 400, WASHINGTON, DC, 20036, US

(21) Appl. No.: 12/068,285

(22) Filed: Feb. 5, 2008

Related U.S. Application Data
(60) Provisional application No. 60/899,410, filed on Feb. 5, 2007.
(60) Provisional application No. 60/900,118, filed on Feb. 8, 2007.
(60) Provisional application No. 60/900,987, filed on Feb. 13, 2007.
(60) Provisional application No. 60/924,083, filed on Apr. 30, 2007.
(60) Provisional application No. 60/924,125, filed on May 1, 2007.
(60) Provisional application No. 61/006,094, filed on Dec. 19, 2007.
(60) Provisional application No. 61/006,095, filed on Dec. 19, 2007.
(60) Provisional application No. 61/006,097, filed on Dec. 19, 2007.
(60) Provisional application No. 61/006,099, filed on Dec. 19, 2007.
(60) Provisional application No. 61/006,100, filed on Dec. 19, 2007.
(60) Provisional application No. 61/006,098, filed on Dec. 19, 2007.

(51) Int. Cl. A61B 5/00; G06Q 50/00

(52) U.S. Cl. 600/300; 705/3

(57) ABSTRACT

Systems, methods and computer program products for a non-invasive, automated method of receiving, collecting, storing and transmitting a person's physiological data and activity levels for the purposes of determining the well-being of a person are disclosed. Aspects of the present invention are also directed to making additional health status determinations based on the historical information and trends of the collected data. Such aspects of the present invention readily lend themselves to incremental component and functionality modifications, which would allow for any number of physiological data to be collected through the introduction of any number of non-invasive sensors. This results in higher accuracy, reliability, and utility of the collected information, further solidifying the uniqueness and desirability of the invention, for consumer and clinical Wireless Body Area Networks (WBAN) applications.

### CROSS REFERENCE TO RELATED APPLICATIONS

This application claims the benefit of, and is related to, the following of Applicants' co-pending applications:

U.S. Provisional Application No. 60/897,243 titled “Method and System for Physiological Data Readings, Transmission, and Presentation,” filed on Jan. 25, 2007;

U.S. Provisional Application No. 60/899,410 titled “Communications and Biosensor Device,” filed on Feb. 5, 2007;

U.S. Provisional Application No. 60/900,118 titled “Device for Non-Invasive Physiological Data Readings,” filed on Feb. 8, 2007;

U.S. Provisional Application No. 60/900,987 titled “Physiological Data Processing Architecture for Situation Awareness,” filed on Feb. 13, 2007;

U.S. Provisional Application No. 60/924,083, titled “Heterogeneous Data Collection and Data Mining Platform,” filed on Apr. 30, 2007;

U.S. Provisional Application No. 60/924,125 titled “Heterogeneous Data Collection and Data Mining Platform” filed on May 1, 2007;

U.S. Provisional Application No. 61/006,094, titled “Improved Communications and Biosensor Device,” filed on Dec. 19, 2007;

U.S. Provisional Application No. 61/006,095, titled “Gateway for Discrete and Continuous Monitoring of Ambient Data with Emergency Functions,” filed on Dec. 19, 2007;

U.S. Provisional Application No. 61/006,097, titled “Gateway for Discrete and Continuous Monitoring of Physiological Data,” filed on Dec. 19, 2007;

U.S. Provisional Application No. 61/006,099, titled “Method and System for Discrete and Continuous Monitoring or Physiological and Ambient Data,” filed on Dec. 19, 2007;

U.S. Provisional Application No. 61/006,100, titled “User Interface for System for Discrete and Continuous Monitoring of Physiological and Ambient Data,” filed on Dec. 19, 2007;

U.S. Provisional Application No. 61/006,098, titled “Method and System for Data Transmission for Use with Biosensor Device or Gateway,” filed on Dec. 19, 2007; and

U.S. Non-provisional application Ser. No. ______, titled “System and Method for Physiological Data Readings, Transmission, and Presentation, filed on Jan. 25, 2008; each of which is incorporated by reference herein in its entirety.

### BACKGROUND OF THE INVENTION

1. Field of the Invention

The present invention generally relates to automated systems and methods for the receipt, collection, storage, transmission and presentation of physiological data. More particularly, the present invention is directed to wireless body area network systems, methods and computer program products for facilitating the receipt, collection, storage, transmission and presentation of such physiological data.

2. Related Art

In today's technological environment, systems containing individual sensors with (or without) wireless transceivers are known and used for collecting and transmitting physiological data (e.g., vital signs such as blood pressure, pulse rate, respiration), which reflect the health status or well-being of a person, as well as ambient or motion data. Such systems are commonly referred to as Body Area Networks (BANs). If the wireless communication is employed the system is called Wireless Body Area Networks (WBANs). The goal of WBANs, and their supporting information infrastructures, is to offer unprecedented opportunities to (remotely) monitor the state of health of the wearer of such systems, without constraining the activities of the wearer. The convergence of technologies such as low-power wireless sensor platforms, power efficient wireless communication standards, innovative sensors, plug-and-play device operation, and off-the-shelf components for low-power sensors, handheld and wearable computers, electronic medical records, and the Internet have allowed WBAN technologies to come about.

WBANs have been used, for example, for monitoring of elderly people and/or other individuals that need frequent monitoring. These individuals may live in a nursing home or other managed care facility environment. Such environments, obviously, limit the monitored individuals' ability to continue living independently (e.g., in their own homes). This is primarily because care givers may not be available to constantly monitor their physiological indicators and/or ambient factors, especially in the case of care givers who do not live in close proximity to the monitored individual. With the use of WBANs, however, one or more sensors of differing types are employed to remotely and ambulatory monitor a user's physiological indicators and/or other ambient signals (e.g., motion sensors, electrocardiograms (ECGs), electromyograms (EMGs), electro-encephalograms (EEGs)). The sensors can be located on the body as wearable apparatuses or tiny intelligent patches, integrated into clothing, or even implanted below the skin or muscles.

WBAN systems also typically utilize a storage device for aggregating the sensed and collected data for future access and processing, or are dependent on smart phones and similar mobile devices for collecting and then transmitting the data to a healthcare provider or a health monitoring entity.

While the above-described systems work for their respective intended purposes, the state of the art is such that these systems or devices are often cumbersome and difficult to operate. This is true both from the perspective of weight and size of the WBAN-related equipment, as well as because many such systems require numerous wires for interconnecting the various components.

Given the foregoing, what are needed are improved wireless, near-real time WBAN systems, methods and computer program products for facilitating the receipt, collection, storage, transmission and presentation of physiological data. There is also a need for an improved lightweight, wireless system that allows for unobtrusive operation, ease of use and desirability.

### BRIEF DESCRIPTION OF THE INVENTION

Aspects of the present invention meet the above-identified needs by providing systems, methods and computer program products for facilitating the receipt, collection, storage, processing, secure transmission, and presentation of physiological data through non-invasive means. Additional aspects of the present invention are capability of two-way voice communication with pre-defined remote location(s).

An advantage of certain aspects of the present invention is that they provide a simple to wear, lightweight system or device, thus making it ideal for everyday use, without impeding the user's normal activities. Aspects of the present invention may, for example, double as a wristwatch or a pendant, having such simplicity of everyday use without impeding normal activities. Most notably, an aspect of the present invention may contain two wireless transceivers, one used for receiving data from a WBAN and the other for transmitting the data to an external device, external system or remote location. This means the present invention described herein is suitable as a wireless WBAN gateway. The WBAN gateway may be configured to be worn as a wristwatch, as a pendant, attached to a belt, placed in clothing such as a pocket, etc.

Another advantage of aspects of the present invention is that a user interface is provided, such that a user may set and change information related to the monitored individual, such as pre-programmed emergency telephone numbers, contact information in case of an emergency and the like.

Another advantage of certain aspects of the present invention is that it is completely wireless and the sensed and collected physiological data and/or ambient data are made available in near real-time, both through a secure browser connection and on mobile devices, to service subscribers.

Further features and advantages of certain aspects of the present invention, as well as the structure and operation of these various aspects of the present invention, are described in detail below with reference to the accompanying drawings.

### BRIEF DESCRIPTION OF THE DRAWINGS

The features and advantages of certain aspects of the present invention will become more apparent from the detailed description set forth below when taken in conjunction with the claims and drawings, in which like reference numbers indicate identical or functionally similar elements. Additionally, the left-most digit of a reference number identifies the drawing in which the reference number first appears.

FIG. 1 is an electronic block diagram illustrating an exemplary device according to an aspect of the present invention.

FIG. 2 is a block diagram of an exemplary computer system useful for implementing aspects of the present invention.

FIG. 3 is a flowchart depicting the operation and data flow of a system device according to an exemplary aspect of the present invention.

FIG. 4 is a flow chart depicting the system device voice data flow according to an exemplary aspect of the present invention.

FIG. 5 is a diagram of an exemplary system for implementing aspects of the present invention.

### DETAILED DESCRIPTION

Aspects of the present invention are directed to systems, methods and computer program products for facilitating the receipt, collection, storage, transmission and presentation of physiological data.

In an aspect of the present invention, an integrated system for obtaining a person's physiological and/or ambient data (e.g., vital signs), through non-invasive methods, securely transmitting the information, and transforming the information into an easily-understood display is disclosed. That is, a physiological and activity data aggregation, transmission and presentation system, method and computer program product for the purpose of monitoring a person's vital signs by the person's family members, care takers, healthcare providers and the like, through non-invasive features are disclosed. Such a system, in one aspect, includes one or more physiological sensors, with each sensor being adapted or capable of being attached to a human body, a computer system, which communicates with the sensors, one or more short- and long-range transceivers, software for data receipt, collection, storage, aggregation and transmission from the one or more sensors.

The system, in an additional aspect, may include software for the retrieval, manipulation, analysis, display and transmission of the physiological data to an end user or a remote location which may be pre-defined. This disclosed system may be completely wireless and could present the data to end users at remote locations on a near-real-time basis. Furthermore, the system components placed on a person's body may be small and lightweight, so that these components do not interfere with the user's normal daily activities. Finally, the system and device offers an alert button for emergency two-way voice communication.

In another aspect of the present invention, the system includes a mechanical device for sensing human orientation and activity. This mechanical device, such as an accelerometer or an inclinometer or the like, may be used to determine the state of the user—whether the user is moving fast or slow, whether the user has fallen, current body position, etc.

In another aspect of the present invention, the system includes a clock, position locator and a microphone. The position locator is used to determine the physical location of the user. This could be done through the use of the global positioning system (GPS) or through proximity and triangulation with known devices that may be configured, within the person's environment, just for this purpose.

In another aspect of the present invention, the computer system includes a processor and a display interface for displaying the information or data gathered on a display unit. The computer system also includes one or more memory component, which may be random access rewritable memory (RAM), where the memory component is in operative communication with the processor. In an additional aspect, the computer system includes a secondary removable storage unit. The computer system also includes a communications interface which is also in operative communication with the processor.

In another aspect of the present invention, the method and computer program product perform the steps of receiving physiological data indicative of the health status of a person from one or more sensors, the sensors being capable of being attached to the body of a person, storing the physiological data received from the one or more sensors, processing the physiological data received and transmitting the raw or processed physiological data. The receipt and transmission of the physiological data is primarily done by the transceivers.

In an additional aspect, the method and computer program product perform the steps of compressing the physiological data and preparing the data for later transmission.

In an additional aspect, the method and computer program product perform the step of encrypting the physiological data. The encryption is performed in order to secure the physiological data during transmission.

In an additional aspect, the method and computer program product perform the step of sensing physical and orientation activity using a mechanical device, which may be an accelerometer, an inclinometer or the like. Additional steps performed by the method and computer program product include the steps of generating data regarding the physical and orientation activity and analyzing the generated data.

In an additional aspect, the method and computer program product perform the step of locating a wireless system. This could be done through the use of the global positioning system (GPS) or through proximity and triangulation with known devices that may be configured, within the person's environment, just for this purpose.

In an additional aspect, the method and computer program product perform the steps of synchronizing the internal components of the wireless system and displaying reminders and warnings. Such reminders may include those that remind the user to change the batteries used to power the system and device, or to wam personnel monitoring a certain user about the state of that user.

In yet another aspect, the method and computer program product perform the step of monitoring the power level of the wireless system. This may be done by first determining whether the system has reached a certain power threshold and then generating an audible notification when the wireless system has reached a certain power threshold. The audible notification warns the user or personnel that the remaining power of the system is low.

In yet another aspect, the method and computer program product perform the steps of managing communications to and from the system and interpreting incoming communications to the system. This may be done using the computer system's processor.

Aspects of the present invention will now be described in more detail herein in terms of the above exemplary context and the accompanying figures. This description is for convenience only and is not intended to limit the application of aspects of the present invention. In fact, after reading the following description, it will be apparent to those skilled in the relevant art(s) how to implement aspects of the following invention in alternative ways.

The terms “person,” “patient,” “subject,” “user,” “subscriber,” “client,” “wearer,” “being,” and/or the plural form of these terms are sometimes used interchangeably herein to refer to those person(s) or other living being(s) from whom physiological data are being collected (or, in some cases, the safety and medical personnel and professionals entrusted with their well being), and thus would benefit from the system, method and computer program products that aspects of the present invention provide for facilitating the receipt, collection, storage, transmission and presentation of physiological data of persons or other living beings.

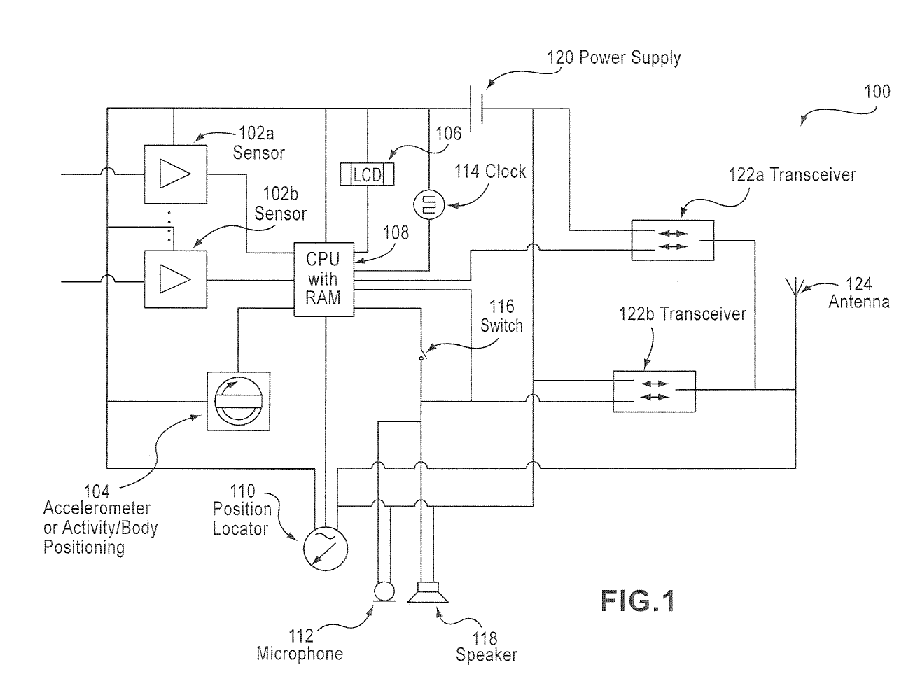

Referring to FIG. 1, an electronic block diagram of device 100 is shown according to an aspect of the present invention. In such an aspect, device 100 comprises of one or more sensors 102a and 102b, an accelerometer or inclinometer or activity/body positioning device 104, a liquid crystal display (LCD) 106, an on board computer 108 with memory, a position locator 110, a microphone 112, a clock 114, a switch 116, a speaker 118, a power supply 120, short- and long-range transceivers 122a and 122b and antenna 124.

Sensors 102a and 102b are used for collecting physiological and activity data. Physiological data collected by sensors 102a and 102b may include body surface temperature, ambient temperature, heart rate, ECG, orientation, galvanic skin resistance, photoplethysmograph, location, activity, etc. Sensors 102a and 102b are electronic devices which receive signal impulses from electrodes or sensing surfaces. The electrodes or sensing surfaces may or may not touch on the surface of the skin. These electrodes transmit their electrical signals to the sensors, which in turn interpret the signals and forward the data to on-board computer 108.

Accelerometer, inclinometer or similar mechanical device 104 is used to sense the orientation and physical activity of the person wearing device 100. Accelerometer 104 can provide data to determine the state of the person—for example whether the person is moving fast or slow, being immobile or resting—but can also be used to provide information on the person's orientation, for example sitting, lying down, a sudden change of body position that has occurred (e.g. sudden fall), etc. Accelerometer data is first sent to on-board computer 108 and is then analyzed using computer programs (algorithms) resident on on-board computer 108. The computer programs are configured to determine the user's condition (e.g., whether the user has fallen).

Position locator 110 is used to determine the physical location of the person wearing device 100. As will be appreciated by those skilled in the relevant art(s), this may be done through the use of the global positioning system (GPS) or through proximity and triangulation, for example cellular triangulation, etc., with known devices that may have been configured, within the person's environment, just for this purpose. Once the user's location has been determined, the location data is sent to on-board computer 108 for analysis and storage. The location feature may be optional and may be provided only at predetermined times such as after an emergency has been identified.

Clock 114 is primarily utilized to synchronize the various components found on device 100. Because there is communication between device 100 and other external devices, through transceivers 122, clock 114 is also used for the synchronization of such external devices. Device 100 and communications synchronization is managed through on-board computer 108. Another function of clock 114 is for displaying the current time on LCD 106 of device 100. This gives device 100 the feel of a convention digital wristwatch used for telling time and displaying reminders and warnings.

Transceivers 122a and 122b and antenna 124 present the communication interface for device 100 to communicate with external devices within a WBAN. One or more antennas (124) are used for transmitting and receiving signals while transceivers 122a and 122b define the communication protocols and frequencies supported for communication. Any number of protocols may be used, the majority of which specify an operating frequency range. Other protocols may operate on a single frequency. Transmission protocols may include ZigBee (802.15.4), Cellular (CDMA, GSM and others), Wireless (802.11a/b/g/n), Wi-Fi (802.11x), ANT, Bluetooth (802.15.1), and Ultra Wide Band (UWB). In one aspect, device 100 may have two transceivers, one for short-range communications and another for long-range communications. In another aspect of the present invention, an interface for connection to wired intelligent sensors is also possible. Short-range transceiver 122a is used for communication with other devices which are located in close proximity with the device, usually within three to six feet. Such other devices, in one aspect of the present invention, may be a body patch comprised of sensors collecting other physiological data different than, and in addition to, device 100 as described in more detail in co-pending U.S. Provisional Application No. 60/900,118 titled “Body Patch for Non-Invasive Physiological Data Readings,” filed on Feb. 8, 2007, which is incorporated by reference herein in its entirety. Long-range transceiver 122b is used for communication with remote devices and individuals, through pre-established wireless and wired communications networks.

Microphone 112 is used for audio communication between the user and an individual at a remote location. Thus, microphone 112 captures sounds, which in turn are sent to on-board computer 108 for initial processing and then transmitted through long-range transceiver 122b to a pre-defined remote location. Similarly, speaker 118 plays back sounds transmitted by the remote individual or from the remote location. This enables the person wearing device 100 to establish two-way voice communication with an individual at the pre-defined (remote) location.

Duplex voice communication between a user and a remote individual or location is made possible by switch 116. Switch 116 is accessible from the surface of device 100 and is triggered by the person wearing it. Once triggered, switch 116 opens and closes an electronic circuit, which then activates long-range transceiver 122b and establishes on-demand duplex voice communication.

Power supply 120 may consist of one or multiple batteries, which, depending on the configuration, may or may not be rechargeable. Power supply 120 provides the necessary electrical power to the electronic components, so they can operate properly and perform their respective intended functions. Because the availability of sufficient power is necessary for the proper operation of the electronic components within device 100, on-board computer 108, in one aspect of the present invention, monitors the power levels and generates an audible and/or visual notification when it detects power levels below a certain threshold. This power threshold is defined by the suite of electronic components on device 100 and their collective power requirements for proper operation. The audible signal informs the wearer that the power level on device 100 is becoming insufficient for sustaining ongoing operations.

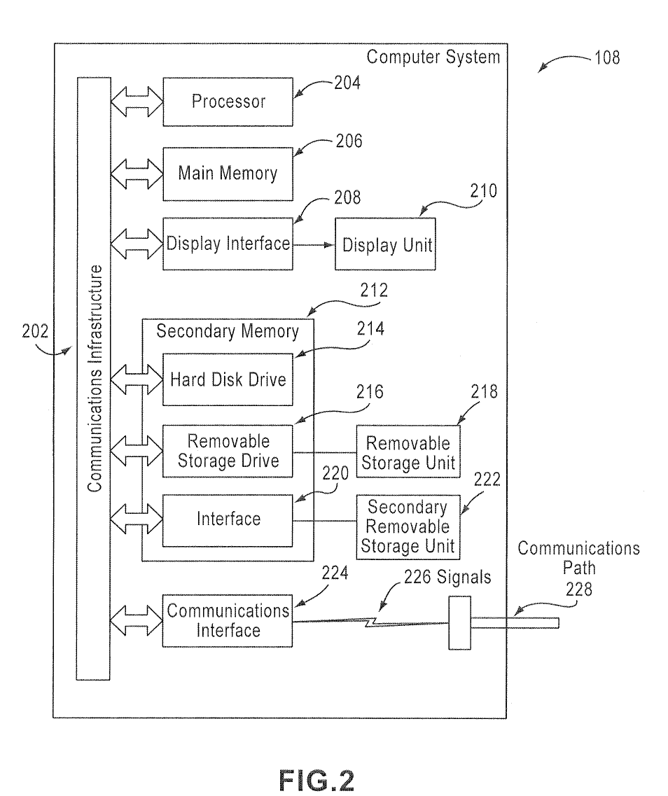

On-board computer 108 is the controlling unit for all electronic components within device 100 and the processing unit for all the signals and data. On-board computer 108 also manages all of device 100 communications with external devices, through transceivers 122a and 122b. In effect, on-board computer 108 is a computer system similar to the block diagram shown in FIG. 2.

Referring now to FIG. 2, a computer system 108 depicting various computer system components for use with an exemplary implementation of a data collection, communications and analysis device 100, in accordance with an aspect of the present invention, is shown.

Various software aspects are described in terms of this exemplary computer system. After reading this description, it will become apparent to a person skilled in the relevant art(s) how to implement the invention using other computer systems and/or architectures.

The computer system 108 includes one or more processors, such as processor 204. Processor 204 is connected to a communications infrastructure 202 (e.g., a communications bus, cross-over bar or network). Computer system 108 can include a display interface 208 that forwards graphics, text and other data from the communication infrastructure 202 (or from a frame buffer not shown) for display on the display unit 210.

Computer system 108 also includes a main memory 206, preferably random access memory (RAM), and may also include a secondary memory 212. The secondary memory 212 may include, for example, a hard disk drive 214 and/or a removable storage drive 216, representing a flash memory card, a magnetic tape drive, an optical disk drive, etc. The removable storage drive 216 reads from and/or writes to a removable storage unit 218 in a well known manner. Removable storage unit 218 represents a flash memory card, magnetic tape, optical disk, etc. which is read by and written to by removable storage drive 216. As will be appreciated, the removable storage unit 218 includes a computer usable storage medium having stored therein computer software and/or data.

In alternative aspects, secondary memory 212 may include other similar devices for allowing computer programs or other instructions to be loaded into computer system 108. Such devices may include, for example, a secondary removable storage unit 222 and an interface 220. Examples of such may include a program cartridge and cartridge interface (such as that found in video game devices), a removable memory chip (such as an flash memory, erasable programmable read only memory (EPROM), or programmable read only memory (PROM)) and associated socket, and other secondary removable storage units 222 and interfaces 220, which allow software and data to be transferred from the secondary removable storage unit 222 to computer system 108.

Aspects of the present invention may be implemented using, for example, a microcontroller. A microcontroller may include a microprocessor on a single integrated circuit configured to operate as a single-chip embedded system. The microcontroller may include a CPU, RAM as working memory, program memory (e.g. flash memory, ROM, or PROM), direct memory access (DMA), timers, I/O ports, other serial communications interfaces, EEPROM or flash memory for permanent data storages, peripheral devices (such as timers, event counters, etc.), a clock generator, analog-to-digital converters, digital-to-analog converters, comparators, and in-circuit programming and debugging support.

Computer system 108 may also include a communications interface 224. Communications interface 224 allows software and data to be transferred between computer system 108 and external devices. Examples of communications interface 224 may include a modem, a serial interface, such as Universal Serial Bus (USB) or RS232, a network interface (such as an Ethernet card), a communications port, etc. Software and data transferred via communications interface 224 are in the form of signals 226 which may be electronic, electromagnetic, optical or other signals capable of being received by communications interface 224. These signals 226 are provided to communications interface 224 via a communications path (e.g., channel) 228. This channel 228 carries signals 226 and may be implemented using wire or cable, fiber optics, a telephone line, a cellular link, an radio frequency (RF) link and other communications channels.

In this document, the terms “computer program medium” and “computer usable medium” are used to generally refer to media such as removable storage drive 216, a hard disk installed in hard disk drive 214, and signals 226. These computer program products provide software to computer system 108. The invention is directed to such computer program products.

Computer programs (also referred to as computer control logic) are stored in main memory 206 and/or secondary memory 212. Computer programs may also be received via communications interface 224. Such computer programs, when executed, enable the computer system 108 to perform the features of the present invention, as discussed herein. In particular, the computer programs, when executed, enable the processor 204 to perform the features of the present invention. Accordingly, such computer programs represent controllers of the computer system 108.

In an aspect where the invention is implemented using software, the software may be stored in a computer program product and loaded into computer system 108 using removable storage drive 216, hard drive 214 or communications interface 224. The control logic (software), when executed by the processor 204, causes the processor 204 to perform the functions of the invention as described herein.

In another aspect, the invention is implemented primarily in hardware using, for example, hardware components such as application specific integrated circuits (ASICs). Implementation of the hardware state machine so as to perform the functions described herein will be apparent to persons skilled in the relevant art(s). In yet another aspect, the invention is implemented using a combination of both hardware and software.

Electronic components of device 100 are packaged in a water tight container to prevent short circuits from moisture. The electronic components are molded on a printed circuit board (PCB), which may be constructed of flexible material. Antenna 124 is internal to device 100 and is located either on the PCB or within the bracelet of device 100.

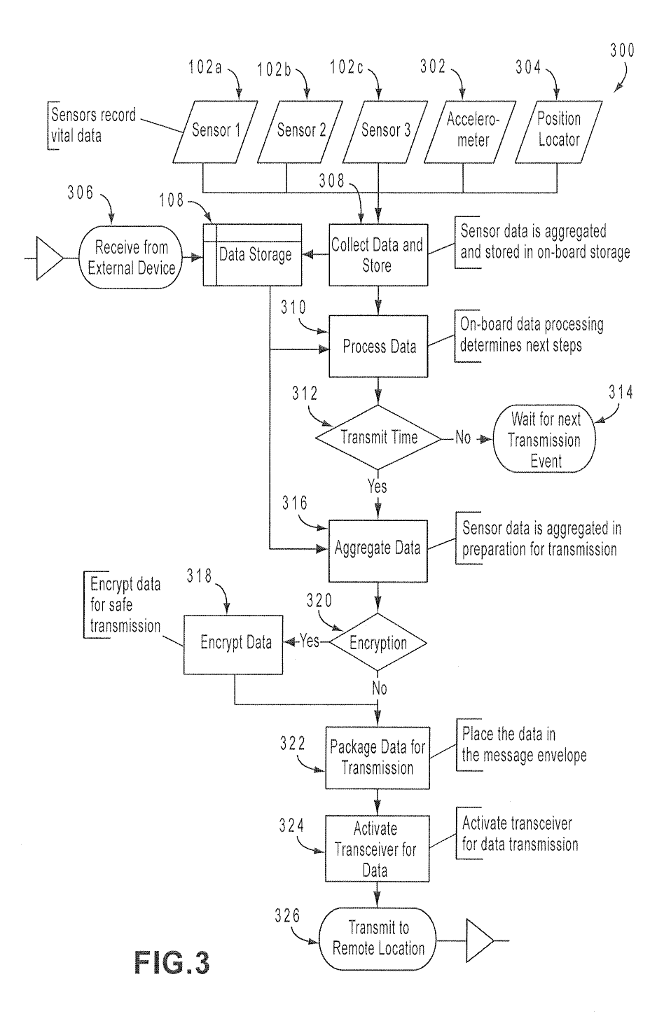

Referring to FIG. 3, a flowchart depicting exemplary operation and data flow 300 of the device 100 of according to an aspect of the present invention is shown. In this aspect, data collected and generated by accelerometer 302 and position locator 304, along with physiological and/or ambient data received in step 306 by one of or a combination of sensors 102a, 102b and 102c, contained within device 100, are collected and stored in the internal storage of on board computer 108 in step 308 to determine patterns and compress the data. In step 310, the data is processed for on-device analysis. Next, in step 312, on-board computer 108 determines whether it is time to initiate a scheduled transmission to a pre-defined remote location. If it is not yet time for the transmission, device 100 waits in step 314 until it is time to transmit. If in step 312 on-board computer 108 determines that it is time to transmit, the transmission preparation process begins. In preparing the data for transmission, all the data from the internal storage of on board computer 108 (including the data from any external devices and readings from the sensors 202a-c) are aggregated and compressed in step 316.

In step 320, processor 108 will determine whether to encrypt the aggregated data or not. If processor 108 determines that the data should be encrypted, the data is then encrypted in step 318. Otherwise, the data is packaged for transmission in step 322 in an unencrypted manner. After encryption of the data in step 320, the data is then constructed, including identifying information, destination, transmission type and other pertinent information in step 322 in preparation for transmission. The data is then packaged into a message, according to the (long-range) transmission protocol being employed. Any number of protocols may be used, the majority of which specify an operating frequency range. Other protocols may operate on a single frequency. In alternate aspects, transmission protocols may include ZigBee (802.15.4), Cellular (CDMA, TDMA, GSM and others), Wireless (802.11a/b/g/n), Wi-Fi (802.11p), ANT, Bluetooth (802.15.1), or custom wireless protocols working in any available frequency or frequencies. In step 324, transceiver 122b is activated. Finally, after transceiver 122b is activated and a network connection is established in step 326, a burst transmission of data from device 100 to a pre-defined remote location occurs, and transceiver 122b is then deactivated until the next transmission event (e.g., until data flow 300 is repeated). At the remote location, the transmitted data may undergo further analysis, processing and preparation for reporting as described in more detail in U.S. Provisional Application No. 60/897,243 titled “Method and System for Physiological Data Readings, Transmission, and Presentation,” filed on Jan. 25, 2007, which is incorporated by reference herein in its entirety.

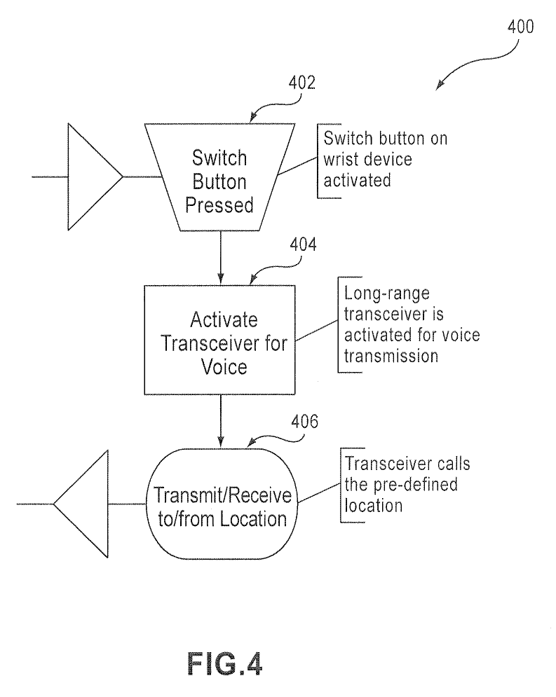

Referring to FIG. 4, a flowchart depicting exemplary voice operation and flow 400 of device 100 according to an aspect of the present invention is shown.

Data flow 400 is initiated by a person wearing device 100 by pressing button or switch 116 in step 402. This activates log-range transceiver 122b in step 404 which establishes connection with a pre-defined remote location, normally a call center which receives such calls. Two-way voice communication between the call center or remote location and the user with device 100 is then established in step 406. The communication could be indicative of an alert condition, requiring immediate attention by another human being such as health-care providers, emergency first responders, call center personnel, etc.

If there are multiple pre-defined locations, one could automatically be selected, prior to establishing the connection. The presence of these communication capabilities makes it possible for an individual at a remote location to page the wearer of device 100, thus establishing reverse two-way voice communication.

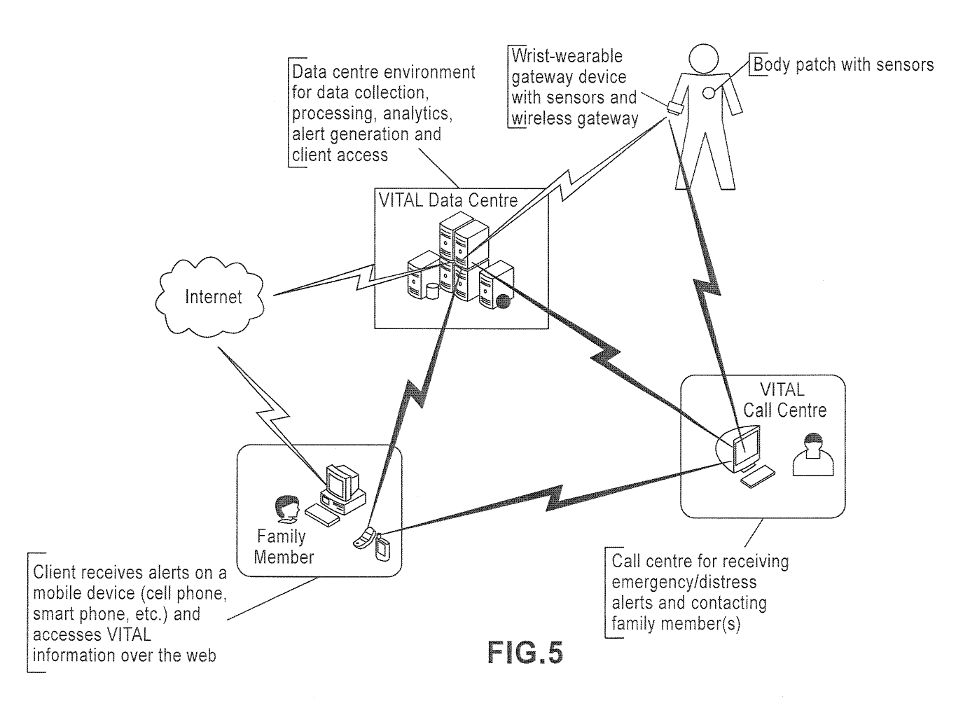

FIG. 5 provides a high-level depiction of an embodiment of the overall system and points of communication between the various components.

An embodiment of the present invention depicted in FIG. 5 includes physiological sensors, a gateway device, short and long range transceivers, proprietary software for data aggregation and transmission from multiple sensors, a data centre environment with multiple computers and custom software for data storage, retrieval, manipulation, analysis, display and transmission to an end-user viewing device via the Internet. This system is completely wireless and presents the data to the end users on both a periodic and optionally, on a real-time basis based on a triggering event. The system components placed on a person's body are small and lightweight so that they do not interfere with normal daily activities. The gateway device offers alert features including simple two-way voice communication.

As discussed above, a sensor may include an adhesive patch attached to the body of a person, integrating several miniaturized physiological sensors. This patch may include a microprocessor, a short-range wireless transceiver and a power supply miniaturized onto a single board. The sensors obtain vital sign physiological data, which are processed, encrypted and aggregated by the microprocessor for transmission by the transceiver to the gateway, at set intervals. Transmissions may be also initiated by the gateway or from the data center.

In accordance with one embodiment, the gateway device is a key feature in the system. The gateway device is a unit that can be comfortably worn by a monitored person. For example, in one embodiment, the gateway may be attached around the wrist and have an appearance similar to a wristwatch. In another embodiment, the gateway may be a pendant worn around the neck of the monitored person. Other embodiments of the gateway are possible such as a unit worn on a belt or placed in a pocket of the monitored person. The gateway is light enough to be comfortably worn by the user and has an ergonomically pleasing structure.

The gateway includes a microprocessor, a short range wireless transceiver, a long-range wireless transceiver, and a power supply. In another embodiment, the gateway may further include additional sensors. The gateway may further include an emergency call button configured to initiate a two-way voice call to an emergency call center. The gateway may also include a plurality of call buttons, wherein each button is configured to initiate a two-way voice call to a pre-programmed telephone number. For example, the gateway may include two such call buttons. The gateway may also include a hidden reset switch that might be used to reset and reinitialize the gateway. The same function might be performed by a combination of existing switches described above.

The pre-programmed telephone numbers and the emergency call number to which the two-way voice calls will be directed may be entered and changed at will by a subscriber at a web interface and long distance communication between the data center and a gateway.

The emergency call button and the pre-programmed call buttons provide the monitored person with the ability to simply initiate voice call without being required to remember and enter a telephone number. In addition, the ability to enter and update the telephone numbers for the pre-programmed call buttons at a web interface allows a monitored person to enter and update information at their convenience which they can draw upon quickly in an emergency or in a time of forgetfulness. In addition, this ability allows a monitored person's family members, care givers, and physicians to update contact information even at a distance from the monitored person. As these numbers can be updated at will, a distant family member can make sure that the monitored person always has current contact information for them. Also, the emergency call number can be updated, if desired, depending upon the location of the monitored person.

The gateway device may include sensors that collect physiological or other data. For example, sensors on the gateway device may include at least one of a sensor for measuring ambient temperature and a sensor for measuring ambient humidity.

In one embodiment, the gateway device processes and encrypts sensor data. The microprocessor in the gateway packages the data for periodic burst transmission through the long-range transceiver, at set intervals.

In another embodiment, the gateway may process the data to determine whether an “event” has occurred. An event may include at least one of the monitored physiological or other characteristics falling outside a predetermined range, or a trend of parameter change specified by the user (e.g. evident trend of heart rate decrease, predefined changes in heart rhythm, etc.). The predetermined range may be set at will through a web interface. The predetermined range information will be sent to the gateway and the sensor, stored, and used in analyzing sensor data for events.

When the gateway determines that an event has occurred, the gateway transmits data in an event mode for a predetermined period of time. Event mode transmission involves a transmission of physiological or other data in a different form that the previously processed, packaged, and periodically transmitted data. For example, in one embodiment, once an event has been identified, the sensor may transmit unprocessed data from at least one sensor on a real time basis in addition to the results of processing. This at least one sensor may be the sensor that recorded the event data that falls outside the predetermined range.

For example, one of the sensors may measure the pulse rate of the monitored person. A family member, care giver, or physician may have set a predetermined acceptable range for the heart rate of the monitored person. If the gateway determines that the heart rate has fallen below the acceptable range, the gateway will begin to transmit data on a different basis. For example, the gateway may transmit the ECG signal or the sequence of RR intervals on a real time basis for a predetermined period of time. Although in this example the gateway transmits real time heart rate data, the gateway may transmit any type of additional data in this event mode, such as body position and activity signals.

In one embodiment, based on the event mode transmission, a number of actions may occur. An emergency call may be placed to the gateway to attempt to contact the monitored person. This call may provide more information on the status of that person or provide assistance to the person. A call may be directed to emergency services to direct an emergency response to the location of the monitored person. A call may be directed to a selected contact such as a family member, a care giver, or a physician. An alarm may be sounded at the gateway device. An emergency message may be sent via e-mail, text message, pre-recorded voice mail, or any other desired method, to a family member, care giver, or physician.

By determining an event, and by sending data in a special event mode transmission, the gateway may provide for automatic alerts or emergency responses in potentially life threatening situations.

After the event mode transmission, the gateway device may return to normal acquisition and transmission of data until another event mode is determined.

Although the gateway may function together with a plurality of additional sensors, the gateway does not require an additional sensor in order to function accurately. For example, the gateway may receive short range transmissions of data from a plurality of sensors placed on the body of the monitored person. The gateway may be capable of accumulating, processing, and packaging this information, together with any information gathered at the gateway device, and transmitting the packaged data

However, in accordance with one embodiment, the gateway device is also capable of functioning properly without data transmissions from additional sensors. In an exemplary embodiment, a subscriber to the system, whether the monitored person, the person's family member, care giver, or physician, may select a “gateway only” option, where the monitored person does not wear any additional sensors. In another exemplary embodiment, although the subscriber to the service may have selected an option with at least one additional sensor, the sensor may function improperly or cease to function for any number of reasons. When this occurs, the gateway is configured to continue to properly collect and transmit data without the sensor.

The remote monitoring system is capable of collecting and analyzing data from a plurality of gateways, whether or not used in connection with at least one additional sensor, at the same time.

The system is also capable of collecting and analyzing data from various types of gateways. For example, the system is capable of functioning with gateways from a plurality of manufacturers. The gateways may include a common interface to the data collection center. This interface may ensure that data transmitted to the data collection center is transmitted in a particular format. Each of the manufacturers may offer more than one model of gateway, with each model offering a different combination of features. The system is capable of identifying the manufacturer and model of gateway from the data transmitted to the remote collection center. This feature provides the ability to make an analysis of the performance of different models and manufacturers of gateway devices.

The gateway may also include an emergency contact tag in a format accessible by first responders. In an embodiment, the emergency contact tag may include at least one of information on the monitored person's medical insurance, primary physician, medications, conditions, and an emergency contact. The format for the emergency contact tag and the unique ID and physician information may be printed or electronic, such as on a smart chip. In another embodiment, in order to protect the privacy of the monitored individual, the emergency contact tag may include only physician contact information and a unique identifier. In this embodiment, the physician will be able to identify the person being monitored based on the unique identifier. In another embodiment, the said information might be accessed through a secure short range communication, or a secure RFID communication or accessing a unique RFID identification pin that may facilitate access to a data center.

In addition, first responders are more readily able to collect information essential to emergency treatment of an unconscious monitored person based on the emergency call button and the pre-programmed call buttons located on the gateway.

While various aspects of the present invention have been described above, it should be understood that they have been presented by way of example, and not limitation. It will be apparent to persons skilled in the relevant art(s) that various changes in form and detail can be made therein without departing from the spirit and scope of aspects of the present invention. Thus, aspects of the present invention should not be limited by any of the above described exemplary aspects, but should be defined only in accordance with the following claims and their equivalents.

In addition, it should be understood that the figures in the attachments, which highlight the structure, methodology, functionality and advantages of aspects of the present invention, are presented for example purposes only. Aspects of the present invention are sufficiently flexible and configurable, such that it may be implemented in ways other than that shown in the accompanying figures.

Further, the purpose of the foregoing Abstract is to enable the U.S. Patent and Trademark Office and the public generally, and especially the scientists, engineers and practitioners in the relevant art(s) who are not familiar with patent or legal terms or phraseology, to determine quickly from a cursory inspection the nature and essence of this technical disclosure. The Abstract is not intended to be limiting as to the scope of aspects of the present invention in any way.

What is claimed is:

1. A system for facilitating the receipt, collection, analysis and transmission of physiological data comprising:
a. at least one physiological sensor capable of being attached to a user utilizing the system;
b. at least one computer system, in operative communication with said at least one physiological sensor and wherein said at least one computer system contains data aggregation and transmission software;
c. a short range transceiver, in operative communication with said at least one computer system;
d. a long range transceiver, in operative communication with said at least one computer system;
e. a mechanical device, in operative communication with said at least one computer system, capable of determining the orientation and physical activity of the user; and
f. a position locator for locating said user.

2. The system of claim 1, wherein the mechanical device is one of the following devices: an accelerometer and an inclinometer.

3. The system of claim 1, further comprising a clock for synchronizing all the devices within the system.

4. The system of claim 1, further comprising an antenna in operative communication with said short-range transceiver and said long-range transceiver.

5. The system of claim 1, further comprising a microphone, wherein said microphone enables duplex voice communication with a remote location.

6. The system of claim 1, wherein the system is in wireless communication with a pre-defined remote location.

7. The system of claim 6, wherein said communication with said pre-defined remote location is via a wireless communications protocol selected from a group consisting of Cellular, ZigBee, Wireless (802.11a/b/g/n), Wi-Fi, ANT, Bluetooth, Ultra wide Band, and custom application protocol.

8. A method for receiving, collecting, storing and transmitting human physiological data using a system comprising:
a. receiving physiological data indicative of the health status of a user utilizing a system comprising at least one physiological sensor, wherein said at least one sensor is capable of being attached to the body of said user;
b. analyzing the physiological data received from said at least one physiological sensor by utilizing at least one computer system, wherein said at least one computer system is in operative communication with said at least one physiological sensor;
c. storing the received physiological data on said at least one computer system;
d. processing the physiological data, by said at least one computer system, in preparation for further action on said physiological data; and
e. transmitting, utilizing a long range transceiver, the physiological data to a pre-defined remote location.

9. The method of claim 8, wherein step (d) comprises compressing the physiological data.

10. The method of claim 8, wherein step (d) comprises encrypting the physiological data.

11. The method of claim 8 further comprising:
sensing orientation and physical activity of said user;
generating data regarding the orientation and physical activity of said user; and
analyzing said generated data.

12. The method of claim 8 further comprising identifying the location of said system.

13. The method of claim 8 further comprising synchronizing the internal components of said system.

14. The method of claim 8 further comprising the step of:
monitoring the power level of the electronic components within said system;
determining whether said system has reached a certain power threshold; and
generating an audible notification when said system has reached a certain power threshold.

15. A computer program product comprising a computer usable medium having control logic stored therein for causing a computer to receive, collect, store and transmit physiological data, said control logic comprising:
a. first computer readable program code means for causing the computer to receive physiological data indicative of the health status of a user utilizing a system comprising at least one physiological sensor;
b. second computer readable program code means for causing the computer to analyze the physiological data received from said at least one physiological sensor;
c. third computer readable program code means for causing the computer to store the received physiological data on the computer;
d. fourth computer readable program code means for causing the computer to process the physiological data, thereby producing presentable data; and
e. fifth computer readable program code means for causing the computer to transmit, by way of a long range transceiver, the physiological data to a pre-defined remote location.

16. The computer program product of claim 15, wherein the fourth computer readable program means comprises control logic for compressing the physiological data.

17. The computer program product of claim 15, wherein the fourth computer readable program means comprises control logic for encrypting the physiological data.

18. The computer program product of claim 15, further comprising:
sixth computer readable program code means for causing the computer to sense the orientation and physical activity of said user;
seventh computer readable program code means for causing the computer to generate data regarding the orientation and physical activity of said user; and
eighth computer readable program code means for causing the computer to analyze said generated data.

19. The computer program product of claim 15, further comprising:
sixth computer readable program code means for causing the computer to synchronize the internal components of said system.

20. A device for receiving, collecting, analyzing, and transmitting physiological data of a monitored person, the device configured to be worn by a monitored person, the device comprising:
a short range transceiver, in operative communication with a sensor patch, the sensor patch including at least one physiological sensor and being configured to attach to the body of the monitored person;
a long range transceiver, in operative communication with a remote computer system configured to aggregate and analyze transmissions;
a power supply; and
a computer usable medium having control logic stored therein.

21. The device according to claim 20, wherein the device is configured as a pendant to be worn by the monitored person.

22. The device according to claim 20, further comprising at least one sensor.

23. The device according to claim 22, wherein the at least one sensor includes at least one sensor selected from a group consisting of an accelerometer, an ambient temperature sensor, an ambient humidity sensor, and a physiological sensor.

24. The device according to claim 22, wherein the short range transceiver is configured to receive data from the sensor patch and wherein the long range transceiver is configured to transmit data based on at least one selected from a group consisting of data from the sensor patch and data collected at the device at predetermined time intervals to the remote computer system.

25. The device according to claim 24, wherein the control logic stored in the computer useable medium is configured such that the device determines when an event has occurred by analyzing data from the sensor patch, and if an event has occurred the long range transceiver transmits data on a near real-time basis.

26. The device according to claim 20 further comprising at least one two-way call button.

27. The device according to claim 26, wherein the device comprises at least two two-way call buttons,
wherein a first button initiates a telephone call to an emergency call center, and
wherein a second button initiates a telephone call to a preprogrammed telephone number.

28. The device according to claim 27, wherein the preprogrammed telephone number is programmed by a user at a web interface with the computer system, and wherein the preprogrammed telephone number may be altered by the user.

29. The device according to claim 20, wherein the long range transceiver uses a wireless protocol selected from a group consisting of Cellular, ZigBee, Wireless (802.11a/b/g/n), Wi-Fi, ANT, Bluetooth, Ultra wide Band, and custom wireless protocol.

30. The device according to claim 20, wherein the device is capable of functioning if the sensor ceases to function.

31. The device according to claim 20, further comprising an emergency contact tag.
